(19) United States
(12) Patent Application Publication
(10) Pub. No.: US 2004/0015186 A1
(43) Pub. Date: Jan. 22, 2004

(54) Meniscal repair device and method of use

(76) Inventor: Edward S. Bittar, Indialantic, FL (US)

Correspondence Address: ROGER M. RATHBUN, 13 MARGARITA COURT, HILTON HEAD ISLAND, SC, 29926, US

(21) Appl. No.: 10/199,720

(22) Filed: Jul. 19, 2002

(51) Int. Cl. A61B 17/08

(52) U.S. Cl. 606/215

(57) ABSTRACT

A device for repairing a meniscus of a patient. An introducer having a sharpened distal end is manipulated to pierce the meniscus across a tear therein. A main body is carried within the introducer and has a distal end having a pair of wings that are biased toward an outwardly extending position where the wings form a tee at the distal end of the main body. When the introducer is properly placed, the main body is pushed so that the distal end of the main body emerges from the distal end of the introducer, thereby freeing the wings to spring to their outwardly biased position. The wings hold the outer surface of the torn meniscus and a fixation device is affixed to the main body to hold the inner surface of the meniscus such that the meniscus is sandwiched between the wings and the affixation device to hold the meniscus with the sides of the tear brought together.

### BACKGROUND OF THE INVENTION

The present invention relates to a device for the repair of the soft tissue of a patient, and, more particularly, to a device and method to repair the meniscus of a knee of the patient.

The meniscus is basically a crescent shaped fibrocartilage shock absorber that is located intermediate the femur or thigh bone and the tibia or shin bone and which assists in retaining the bones in place and includes an outside meniscus, that is, the lateral meniscus and the inside meniscus or medial meniscus. The meniscus, when healthy, absorbs the shock between the thigh bone and ankle bone by cushioning and distributing the forces across the joint as well as stabilizing the knee by controlling its rotation.

A rather common injury that can occur to the knee is a tearing of the meniscus and which can occur as a result, for example, of a twisting of the knee. The tear can result in uneven surfaces that irritate the joint and can cause pain and swelling and a catching sensation. The irritation can increase the longer the tear remains in the meniscus and it is therefore necessary to take some action to repair the tear for the wellbeing of the patient.

Accordingly, certain tears of the meniscus can be corrected by surgery, particularly arthroscopic surgery where a fiber optic instrument is utilized to aid the physician in observing the procedure carried out within the patient's knee so as to cause a minimum of trauma and invasion of the knee. With the surgery, a common practice is to suture the torn portion of the meniscus to thereby repair the tear and to reconstitute the meniscus back to its original status.

Obviously, even with an arthroscopic instrument, the operation is a difficult, delicate procedure, requiring great skill since it requires the use of a suture or sutures that cross or span the tear and then a knot formed and tightened to bring the individual strands of the suture together so as to pull the separated sides of the tear together. One device that is used in such procedures is a double needle suture that has two needles joined by the suture strand. Both needles are passed in parallel fashion through the meniscus tissue on one side of the tear and through the meniscus tissue on the other side of the tear. A loop is formed on the surface of the meniscus when both needles have passed through the meniscus. The free ends of the suture are finally knotted together. A device that facilitates the aforedescribed procedure is shown and described in co-pending U.S. patent application Ser. No. 10/000,000, filed Jul. 12, 2002 and entitled “Arthroscopic and Endoscopic Knot Tying Device and Method of Use” by the present applicant and the disclosure of which is incorporated herein by reference.

Thus, even with the device of the aforedescribed patent application, it is quite difficult to carry out the procedure of suturing the meniscus, particularly due to the remote location of the meniscus, and the overall procedure is a delicate procedure to actually position the sutures so as to pass the needles and the suture strand affixed thereto through the meniscus as well as the torn portion of the meniscus at two locations.

Accordingly, it would be advantageous to have a device that can facilitate the aforedescribed medical procedure to repair and reattach a torn portion of a meniscus without the need for sutures to bring the torn portion of the meniscus back together with the main portion of the meniscus and to a better method of carrying out the procedure itself.

### SUMMARY OF THE INVENTION

Accordingly, the present invention relates to a device and a method to reattach a torn portion of a fibrocartilage meniscus of the knee to the outer rim and base of the meniscus and can be used in the deep recesses of the knee under fiberoptic guidance during arthroscopic surgery and will be specifically described as such, however it will be noted that the present device and method can also be used for the reattachment of other tissue to bone or other soft tissue in areas that are not easily accessible.

The repair device of this invention comprises a main body that is generally elongated and which has a distal end and a proximal end. At the distal end of the main body, there are a pair of wings that are pivotally mounted thereto and which move between a retracted position where the wings are closely aligned with the main body and an extended position where the wings extend outwardly laterally forming a tee configuration at the distal end of the main body. The wings are biased toward that extended position. In one embodiment, the main body has a smooth portion just proximal to the distal end and extending inwardly for approximately one centimeter. Further proximal to that smooth portion, there is formed a series of enlarged areas, preferably a series of annular rings that are of a polymeric material and which are circumferentially molded to the main body and spaced generally an equal distance apart from each other. Proximal to the series of rings, the main body is again smooth continuing through that longest portion of the main body to the proximal end thereof.

As an alternate embodiment, the entire main body may be a smooth outer peripheral surface and not have the annular rings.

The present invention also includes a hollow introducer that is used to insert the main body to a desired location within the particular affected area of the patient where the repair of a meniscus is intended to be carried out. The hollow introducer also has a distal end and a proximal end and the distal end is pointed or sharpened so as to be able to pierce the meniscus of the patient in positioning the main body. The main body is intended to be positioned within the interior of the hollow introducer with the wings in their retracted position such that the main body can readily slide within the hollow introducer.

Thus, in use, the hollow introducer, containing the main body with the wings retracted, is used by the physician to be introduced into the joint of the patient where it is passed through the displaced inner portion of the meniscus first and then through the outer base of the meniscus, thereby passing across a tear in that meniscus. Once the introducer has passed through both of those portions, the main body can be advanced to extend out of the distal end of the introducer by means such as a smooth, obturator or plunger. The plunger therefore pushes the main body so that the distal end of the main body emerges from the distal end of the introducer. The wings, therefore, are no longer constrained by the inner surface of the introducer and are free to spring outwardly by the outward bias so as to assume a tee shape at the distal end of the main body.

At this point, the introducer can be removed, leaving the main body in position penetrating the displaced inner portion as well as the outer base of the meniscus with the wings forming the tee deployed and which are located just beyond the outer rim of the base of meniscus. Thus, with the ringed embodiment, the smooth portion of the main body is located within the meniscus itself and the ringed portion is located proximal of the inner surface of the inner rim of the meniscus.

By use of the main body, the wings can engage and be positioned against the outer surface of the outer rim and a fixation device can be affixed to the main body proximal to the inner surface of the inner portion of the meniscus to hold the meniscus together with the sides of the tear held snugly together.

Accordingly, since the outer surface of the torn meniscus has been engaged and is supported by the tee shaped wings, the fixation device is used to engage and hold the inner surface of the inner portion of the torn meniscus to bring the portions of the torn meniscus together and be held together in a tight relationship. As such, the fixation device is secured to the main body so as to be tightly held against the inner surface of the inner portion of the meniscus. In the embodiment where the main body has a plurality of enlarged areas or rings, reapproximation and fixation of the meniscus fragments can be accomplished by use of the ringed portion of the main body. A fixation device, such as a ring, plug, washer or sleeve with a smooth round opening or a stellate-shaped opening can be slid over the proximal end of the main body and progressively slid along the main body over the succession of rings as counter traction is applied to the main body of the meniscus repair device.

Once the fragments of the meniscus have been reapproximated, the fixation device can be affixed at the appropriate ring and the trailing, proximal portion of the main body can be cut away. If the fixation device is not adequately affixed to the main body at the desired location by attaching the fixation device to a particular ring, the fixation device can, as an alternative, be welded to the main body of the meniscus repair device.

With an alternative embodiment, that is, where the main body of the meniscus repair device has a smooth exterior surface along its full length, the fixation device, such as a plug, can be simply welded to the main body of the meniscus repair device once it has been slid into the desired location along the main body. During the welding process, continuous pressure is applied to the fixation device to be welded at the location while counter traction is applied to the main body of the meniscus repair device to maintain approximation of the fragments of the meniscus.

As a further embodiment, there may be two meniscus repair devices with smooth shafts, spaced apart, and which can be used simultaneously to capture a larger segment of a meniscus. In such case, a fixation device having two openings can be used to accommodate the shafts of the two devices, The combination of the two main bodies and the fixation device can be welded together to carry out the repair of a broader segment of the meniscus. The use of the combination of two adjacent meniscus repair devices is desirable where the meniscus tissue is badly damaged, when meniscus tissue quality is poor or when the meniscus tissue is deformed and is not easily held in position.

With any of the aforementioned embodiments, the torn or damaged meniscus can be brought back together and held in the reapproximated status by use of the present meniscus repair device.

These and other features and advantages of the present invention will become more readily apparent during the following detailed description taken in conjunction with the drawings herein.

### BRIEF DESCRIPTION OF THE DRAWINGS

FIG. 1 is side schematic view of the meniscal repair device constructed in accordance with the present invention;

FIG. 2 is a side schematic view of an alternative embodiment of the present invention;

FIG. 3 is an enlarged side view of the distal end of the device of FIG. 2;

FIG. 4 is a top schematic view of the introducer component of the invention;

FIG. 5 is schematic view of the present meniscal repair device in position affixing two portions of a meniscus together;

FIG. 6 is a perspective view of combined meniscal repair devices of the present invention in a preliminary stage in repairing a meniscus;

FIG. 7 is a perspective view of a later step than shown in FIG. 6 in the use of the meniscal repair devices;

FIG. 8 is a perspective view of the use of combined meniscal repair devices showing an alternative embodiment in a preliminary step in repairing a meniscus;

FIG. 9 is a perspective view of a later step than shown in FIG. 8 in the use of the meniscal repair devices;

FIGS. 10A-10D are front views of fixation devices used in conjunction with the present invention.

### DETAILED DESCRIPTION OF THE INVENTION

Referring now to FIG. 1, there is shown a side schematic view of a meniscal repair device 10 constructed in accordance with the present invention. Describing the components individually, the meniscal repair device comprises a main body 12 that fits within an introducer 14 and is slidably retained in the interior of that introducer 14. The other component of the meniscal repair device is an obturator or plunger 16.

Accordingly, the main body 12 has a distal end 18 and a proximal end 20. At the distal end 18 there are a set of two wings 22 that are affixed to the distal end 18 and may be of a polymeric material. The wings 22 are shown in the retracted position where the wings 22 fit tightly against and in alignment with the main longitudinal axis of the main body 12. With the wings 22 in the retracted position, it can be seen that the main body 12 along with the wings 22 fit within the introducer 14 for movement therein. The wings 22 are pivotable about their distal ends 24 affixed to the main body 12 so the free ends 26 are formed that can extend outwardly.

The wings 22 are inherently biased outwardly and, as will be seen, when the wings are not restrained by the inner surface of the introducer 14, the wings 22 will move so as to extend outwardly from the main body 12 to assume a tee configuration with respect to the longitudinal axis of the main body 12. Accordingly, but for the location within the confined space of the introducer 12, the wings 22 would normally be in their extended position extending outwardly from the distal end 18 of the main body 12.

Taking next, the introducer 14, there can be seen that the introducer 14 has a distal end 28 and a proximal end 30 and preferable the distal end 28 is pointed or has a sharp surface 32 so that the introducer 14 can be pushed by the user to pass through the tissue of the meniscus in carrying out the repair of a meniscus as will later be explained.

The introducer 14 also preferably has an external handle 34 so that the physician can grasp and manipulate the introducer 14 by use of the handle to guide the introducer 14 through the particular meniscus in the repair of the meniscus. Otherwise, the introducer 14 is hollow so as to allow the main body 12 to freely move therethrough and is preferably comprised of a plastic material to be disposable, however, the introducer 14 may also be constructed of a metal material and be reusable after sterilization.

The plunger 16 also fits within the interior of the introducer 14 and can move longitudinal therein. The plunger 16 is used to push the main body 12 when desired by the physician and is a hollow shaft 36 that fits over the main body 12. The plunger 16 has a distal end 38 and a proximal end 40. The plunger 16 is dimensioned to encompass and slide over the main body 12 to be moved to a position where the distal end 38 of the plunger can abut against the slightly larger diameter of the free ends 26 of the wings 22.

At the proximal end 40 of the plunger 16, there is a handle 42 that is provided for the use of the physician to manipulate the plunger 16 as will later be explained. The plunger 16 also has indicia 44 along the exterior surface in order to assist the physician in utilizing the plunger 16. In the preferred embodiment the indicia 44 is a series of graduated markings to enable the physician to ascertain the position of the plunger 16 with respect to the introducer 14.

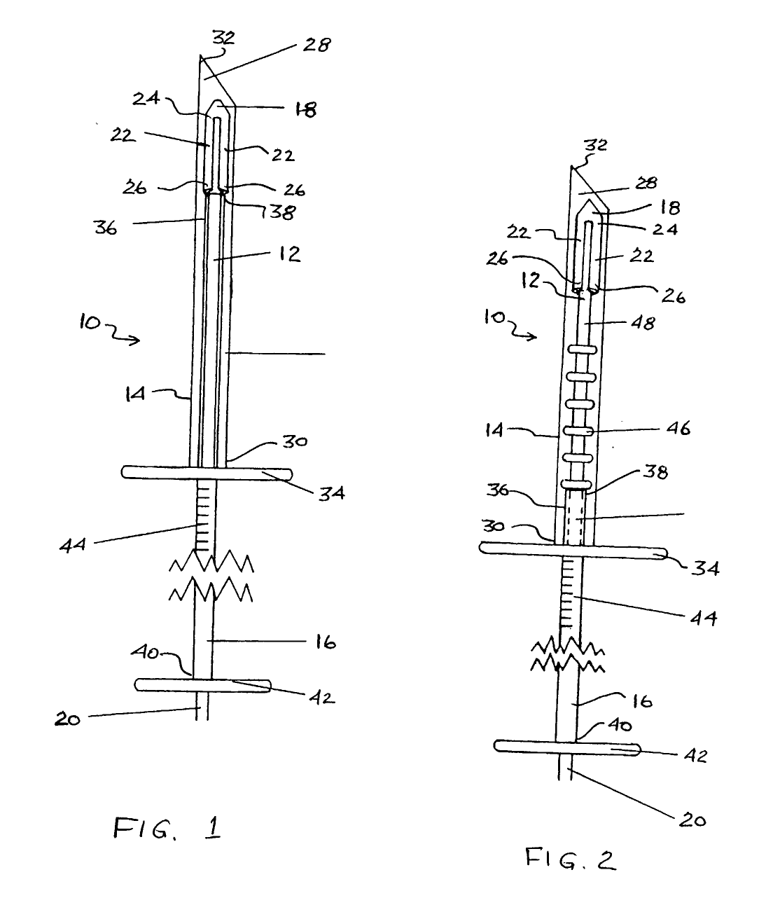

Turning now to FIG. 2, there is shown a side schematic view of an alternative embodiment of the meniscal repair device 10 of FIG. 1 and like numbers have be assigned corresponding components of FIG. 1. As can be seen, however, in FIG. 2, the main body 12 has a series of enlarged portions, normally formed as circumferential rings 46 that extend outwardly from the main body 14 at equal spaced intervals. There is also a smooth portion 48, having no such rings 46 just proximal to the wings 22 and extending inwardly for a predetermined distance, preferably about 1 cm.

Since the rings 46 are present in this embodiment, the distal end 38 of the plunger 16 seats or abuts against the outermost ring rather than being seated at the free ends 26 of the wings 22 as in the FIG. 1 embodiment.

Turning now to FIG. 3, there is shown an enlarged side schematic view of the distal end 28 of the introducer 14 as well as the distal end 18 of the main body 12. As can be seen in FIG. 3, the distal end 18 of the main body 12 has been extended outwardly from the distal end 28 of the introducer 14 so that the wings 22 are no longer constrained to their retracted position as shown in FIGS. 1 and 2 but are free to extend outwardly by means of the inherent bias into a tee configuration at the distal end 18 of the main body 12, that is, the wings 22 assume their extended position.

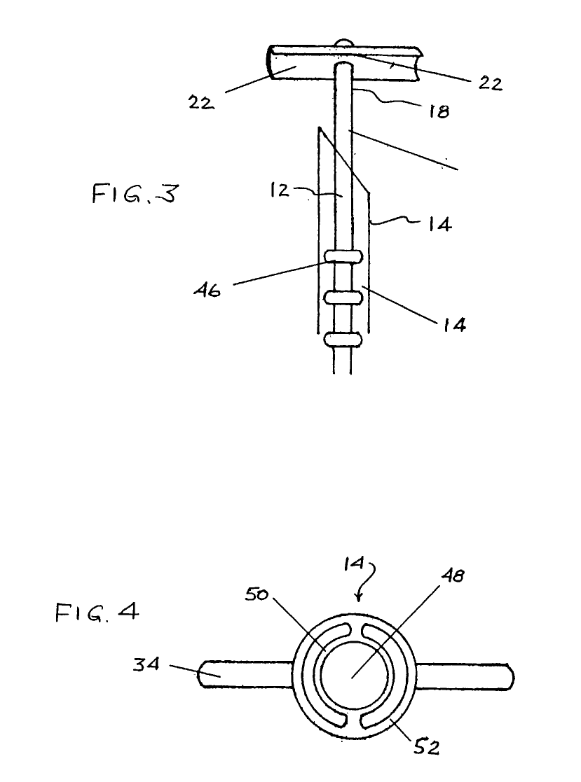

In FIG. 4, there is shown a top view of the introducer 14 used with the invention and showing the central cavity 48 that provides the space for the plunger 16 and the introducer 14 contained within the inner guide 50 that is dimensioned to retain the wings 22 (FIGS. 1-3) in the retracted position. An outer wall 52 completes the introducer along with the handle 34.

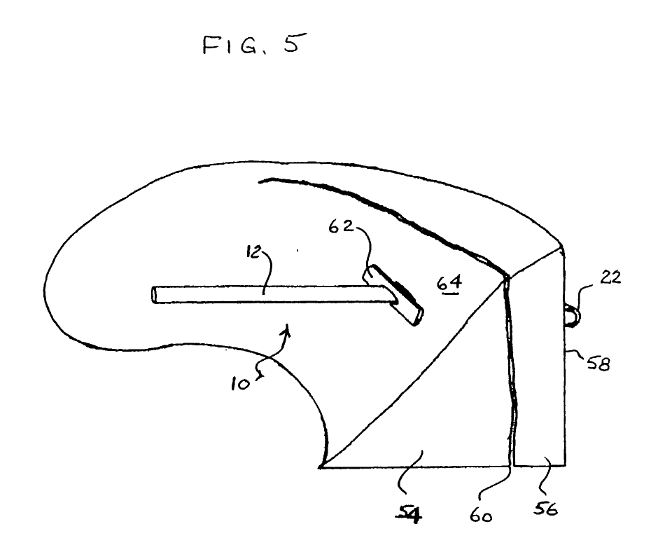

Turning now to FIG. 5 there is shown a perspective view of the present meniscal repair device 10 utilized in the repair or reattachment of a torn portion of a fibrocartilage meniscus of the knee to the outer rim and base of the meniscus and which procedure can be carried out in the deep recesses of the knee under fiberoptic guidance during arthroscopic surgery and will be specifically described as such, however it will be noted that the present device and method can also be used for the reattachment of other tissue to bone of other soft tissue.

In FIG. 5, the main body 12 has been inserted through both the inner portion 54 and the outer rim 56 of the meniscus such that the wings 22 are located tightly against the outer surface 58 of the outer rim. Accordingly the main body 12 has passed across the tear 60 in the meniscus. A fixation device 62 is affixed to the main body 12 in a position where it is tightly drawn against the inner surface 64 of the inner portion 54 such that the entire meniscus is held together, or sandwiched between the wings 22 and the fixation device 62 to bring the outer rim 56 and the inner portion 54 together.

The method of carrying out the present invention can now be explained with reference to FIG. 5 as well as FIG. 1. In the method, the introducer 14 having the main body 12 contained therein, is manipulated by the physician so as to pierce both the inner portion 54 and the outer rim 56 of the meniscus through the employment of the sharp surface 32 at the distal end 28 of the introducer 14. The procedure is facilitated by the physician using the handle 34 of the introducer 14.

Once the introducer 14 has fully pierced both the inner portion 54 and the outer rim 56, the plunger 16 can be grasped by the physician and manipulated to push the main body 12 in the forward direction so that the distal end 18 of the main body 12 can be moved forwardly to emerge from the distal end 28 of the introducer 14 whereupon the wings 22, thereby freed from the constraint of the introducer 14 can spring outwardly by means of the outward bias and thus assume the extended position (see FIG. 3) forming a tee configuration at the distal end 18 of the main body 12.

At that point, the introducer 14 and the plunger 16 can be removed by sliding those components proximally from the main body 12, to leave only the main body 12 in the position piercing the meniscus. The fixation device 62 can then be introduced over the proximal end 20 of the main body and slid along the main body toward the distal end 18 while, at the same time, counter traction is applied to the main body 12 so as to seat both the wings 12 and the fixation device 62 across the meniscus and the meniscus is tightly drawn together. In such position, the fixation device 62 can be affixed firmly to the main body 12 by means such as welding.

Thus with those steps in the method of repairing a meniscus, the completed procedure results in the depiction of FIG. 5, that is, the main body 12 spans the tear 60 and holds the inner portion 54 and the outer rim 56 of the meniscus together.

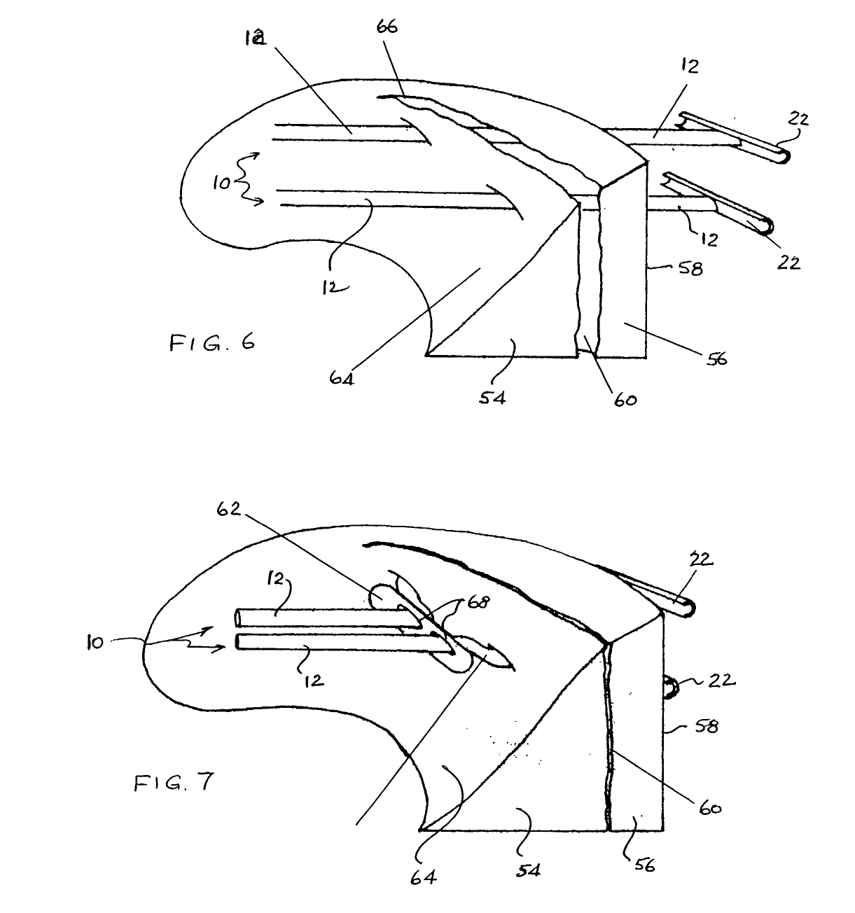

Turning therefore to FIG. 6, there is shown a perspective view of a combination of meniscus repair devices 10. In FIG. 6, two main bodies 12 have been used to pierce the meniscus, one near the apex 66 of the tear 60 and the other displaced outwardly from the apex 66 and both generally parallel to the other. In each case, the wings 22 of the main bodies 12 have moved to their extended positions so as to engage the outer surface 58 of the outer rim 56.

FIG. 7 depicts the further step in the use of the parallel meniscal repair devices of FIG. 6. In FIG. 7, therefore, the wings 22 apply a compressive force to the outer surface 58 of the outer rim 56 and the fixation device 62 has a pair of openings 68, through which the main bodies 12 pass and, again, the fixation device 62 can be affixed to both of the main bodies 12 by means of welding. As with the use of the single meniscal repair device of FIG. 6, the meniscal repair devices 10 of FIG. 7 act to sandwich the meniscus between the wings 22 and the fixation device 62.

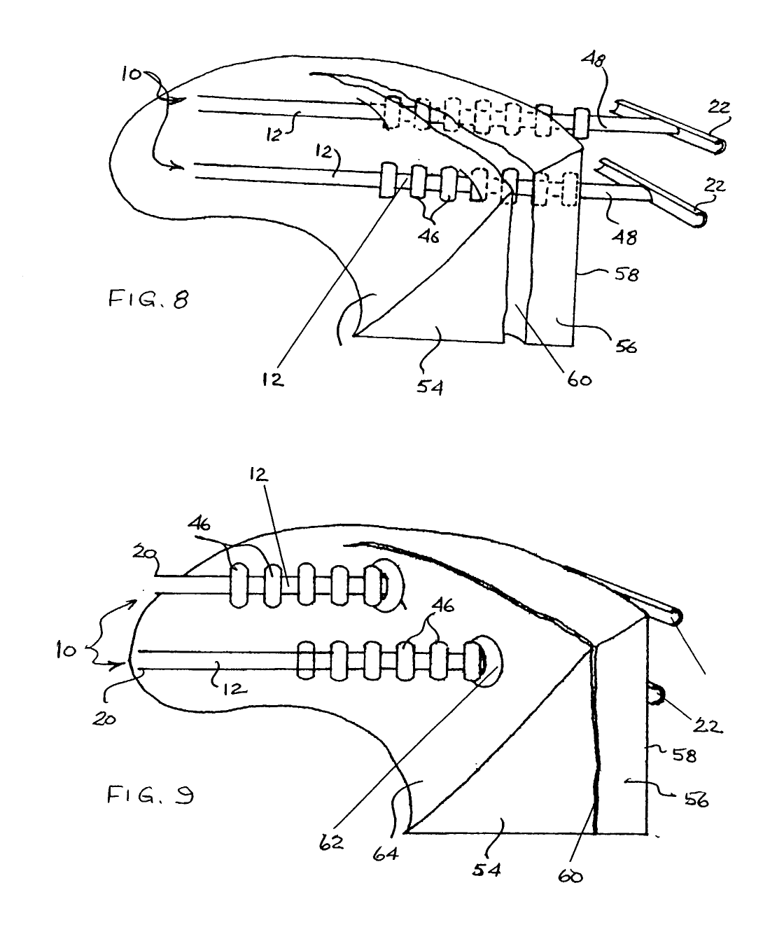

Turning now to FIG. 8, there is shown a perspective view of dual meniscal repair devices 10 of the alternative embodiment of FIG. 2 and where each main body 12 has a plurality of rings 46 that are equally spaced apart. It should be noted in this embodiment that the smooth portion 48 of the main body 12 eventually remains within the meniscus when the outer rim 56 and the inner portion 54 have been brought together in carry out the aforedescribed method.

In FIG. 9, there is a further step in the use of the meniscal repair devices 10 of FIG. 8 wherein the outer rim 56 and the inner portion 54 of the meniscus has been pulled together and the main body 12 secured in place to retain the meniscus with the tear 60 closed. In this embodiment, the fixation devices 62 are rings or washer style devices and are advanced along the main bodies 12 from the proximal ends 20 inwardly and are seated against the inner surface 64 of the inner portion 54 to hold the meniscus in the compressed condition. With the fixation devices 62 of the FIG. 9 embodiment, the fixation device 62 can be advanced over each of the rings 46 until the fixation device 62 reaches the desired ring 46 where it is retained by the ring 46 compressed against the inner surface 64 of the inner portion 54.

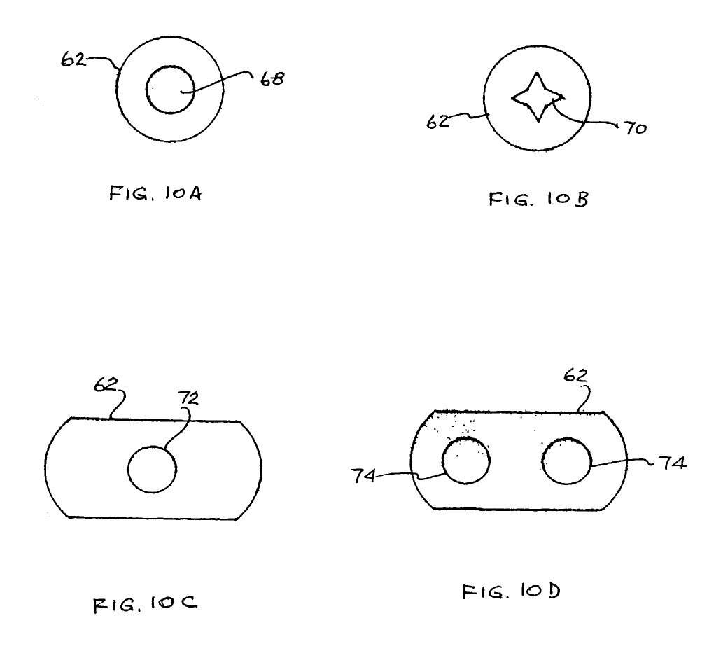

Finally, in FIGS. 10A-10D, there are a series of fixation devices 62 that can be used with the present invention. In FIG. 10A, there is a circular fixation device 62 having a circular opening 68 formed therein and which is therefore usable with the FIG. 2 embodiment or, for that matter, with the FIG. 1 embodiment where the fixation device 62 is welded to the main body 12 (FIG. 1) or affixed thereto by other means. In FIG. 10B, there is a circular fixation device 62 having a stellate opening 70 and which may also be welded to the main body as shown in FIG. 1. but also may be sufficiently affixed to the main body simply by means of the stellate opening 70 grasping the main body 12.

The fixation device 62 of FIG. 10C is an elongated or plate shaped configuration having a circular opening 72 and, with the particular shape shown, can attach more broadly to the meniscus in holding the meniscus in the desired state. Lastly, in FIG. 10D, there is an elongated fixation device 62 having a pair of circular openings 74 and which can be used with the dual meniscal repair devices 10 as shown in FIG. 9, it being seen that the openings 74 can also be of the stellate shape as shown in FIG. 10B.

Those skilled in the art will readily recognize numerous adaptations and modifications which can be made to the meniscal repair device and method of using the same of the present invention which will result in an improved process and device, yet all of which will fall within the scope and spirit of the present invention as defined in the following claims. Accordingly, the invention is to be limited only by the following claims and their equivalents.

I claim:

1. A device for repairing a tear in a patient's tissue to join a first portion of the tissue having an inner surface and a second portion of the tissue having an outer surface, said device comprising a main body having a distal end and a proximal end, at least one wing affixed to the distal end and being movable between a retracted position wherein the at least one wing is closely aligned with said main body and an extended position wherein the at least one wing extends laterally outwardly from said main body, said at least one wing being biased toward said extended position, a hollow introducer having a distal end and a proximal end, said distal end adapted to pierce a first and said second portions of a patient's tissue, said main body adapted to slidingly fit within said introducer with said at least one wing in the retracted position to a position extending beyond said distal end of said introducer wherein said at least one wing can move to the extended position to engage a distal surface of a second portion of tissue, and an affixation device to engage the main body and engage an inner surface of a first portion of tissue.

2. A device as defined in claim 1 wherein said distal end of said introducer is sharpened so as to facilitate piercing a first and second portions of a patient's tissue.

3. A device as defined in claim 1 wherein said at least one wing comprise a plurality of wings.

4. A device as defined in claim 1 wherein said main body comprises at least one enlarged area formed on the main body.

5. A device as defined in claim 4 where said at least one enlarged area formed on the main body comprises a plurality of spaced apart enlarged areas.

6. A device as defined in claim 5 wherein said plurality of enlarged areas are spaced apart beginning a predetermined distance inwardly from said distal end of said main body.

7. A device as defined in claim 5 where in said fixation device is affixed to said main body by affixation to one of said enlarged areas.

8. A device as defined in claim 1 wherein said fixation device is welded to the main body.

9. A device as defined in claim 1 wherein said fixation device has an opening adapted to fit over and slide along the main body wherein the fixation device can be slid along the main body to an inner surface of a first portion of tissue.

10. A device as defined in claim 1 wherein patient's tissue is a meniscus of a knee and a first portion is a portion of a meniscus and a second portion is an outer rim of a meniscus

11. A device for repairing a tear in a patient's tissue, said device comprising a main body having a distal end and a proximal end, at least one wing affixed to the distal end and being movable between a retracted position wherein the at least one wing is closely aligned with said main body and an extended position wherein the at least one wing extends laterally outwardly from said main body, said at least one wing being biased toward said extended position, said main body having a plurality of spaced apart enlarged areas extending outwardly from the main body.

12. A device as defined in claim 11 wherein said main body has a relatively constant dimension from said distal end inwardly a predetermined distance.

13. A device as defined in claim 12 wherein said enlarged areas are rounded, annular bumps formed on the main body.

14. A device for repairing a tear in a patient's tissue, said device comprising a main body having a distal end and a proximal end, at least one wing affixed to the distal end and being movable between a retracted position wherein the at least one wing is closely aligned with said main body and an extended position wherein the at least one wing extends laterally outwardly from said main body, said at least one wing being biased toward said extended position, a fixation device adapted to be affixed to the main body at a predetermined location along said main body.

15. A device as defined in claim 14 wherein said fixation device has an opening adapted to fit over the main body and slide along said main body to said predetermined location.

16. A device as defined in claim 15 wherein said fixation device is welded to said main body at the predetermined location.

17. A method of bringing together an inner portion and an outer rim of a meniscus of a patient's knee, the inner portion having an inner surface and the outer rim having an outer surface, said method comprising the steps of:
providing a hollow introducer having a distal end and a proximal end,
providing a main body having a distal end and a proximal end and at least one wing affixed to the distal end movable between a retracted position where the at least one wing is closely aligned with the main body and an extended position where the at least one wing extends laterally outwardly from the distal end, the at least one wing being biased toward the extended position, the main body being adapted to slidingly interfit within the hollow introducer,
inserting the introducer through the inner portion and the outer rim of the meniscus to pierce the outer surface of the outer rim,
inserting the main body through the introducer with the at least one wing in the retracted position to extend the distal end of the main body from the distal end of the introducer to allow the at least one wing to move to the extended position,
removing the introducer from the meniscus;
manipulating the main body to engage the outer surface of the outer rim and to pull the outer rim and the inner portion closely together,
securing the main body to the inner surface of the inner portion to hold the outer rim and the inner portion in the close together position.

18. A method as defined in claim 17 wherein said step of securing the main body to the inner surface of the inner portion of the meniscus comprises providing a fixation device and advancing the fixation device along the main body until the affixation device contacts the inner surface of the inner portion of the meniscus and securing the fixation device to the main body.

19. A method as defined in claim 18 wherein said step of securing the fixation device to the main body comprises welding the fixation device to the main body.

20. A method as defined in claim 17 wherein said step of providing a main body comprises providing a main body having at least one enlarged area and said step of securing the main body to the inner surface of the inner portion comprises abutting the at least one enlarged area against the inner surface of the inner portion.

21. A method as defined in claim 20 wherein said step of providing a main body comprises providing a main body having a plurality of enlarged areas.

22. A method as defined in claim 21 wherein said step of securing the main body to the inner surface comprises providing a fixation device and securing the fixation device to one of the plurality of enlarged areas.

23. A method as defined in claim 17 where the method further comprises the step of providing a plunger adapted to fit within the introducer and using the plunger to extend the distal end of the main body from the distal end of the introducer.

24. A method as defined in claim 18 wherein said step of providing a main body comprises providing two main bodies and said step of providing an introducer comprises providing two introducers, and said steps of inserting the introducer and inserting the main body comprises inserting both of said introducers and said main bodies and said step for providing a fixation device comprises providing an affixation device having two openings to fit over both of said main bodies.

25. A method as defined in claim 24 wherein said step of securing the fixation device to the main bodies comprises welding the fixation device to both of the main bodies.

26. A method of bringing together an inner portion and an outer rim of a meniscus of a patient's knee, the inner portion having an inner surface and the outer rim having an outer surface, said method comprising the steps of:
providing a main body having a distal end and a proximal end and at least one wing affixed to the distal end and being movable between a retracted position where the wing is closely aligned with the main body and an extended position where the at least one wing extends laterally outwardly from the distal end, the at least one wing being biased toward the extended position,
inserting the main body through the inner portion and the outer rim of the meniscus to pierce the outer surface of the outer rim with the at least one wing in the retracted position to extend the distal end of the main body through the outer surface of the outer rim to allow the at least one wing to move to the extended position,
manipulating the main body to engage the outer surface of the outer rim and to pull the outer rim and the inner portion closely together,
securing the main body to the inner surface of the inner portion to hold the outer rim and the inner portion in the close together position.

27. A method as defined in claim 26 wherein said step of securing the main body to the inner surface of the inner portion of the meniscus comprises advancing a fixation device along the main body until it contacts the inner surface of the inner portion of the meniscus and securing the fixation device to the main body.

28. A method as defined in claim 27 wherein said step of securing the fixation device to the main body comprises welding the fixation device to the main body.

29. A method as defined in claim 26 wherein said step of providing a main body comprises providing a main body having at least one enlarged area and said step of securing the main body to the inner surface of the inner portion comprises abutting the enlarged area against the inner surface of the inner portion.

30. A method as defined in claim 29 wherein said step of providing a main body comprises providing a main body having a plurality of enlarged areas.
